(19) United States
(12) Patent Application Publication
(10) Pub. No.: US 2020/0282031 A1
(43) Pub. Date: Sep. 10, 2020

(54) METHODS AND COMPOSITIONS FOR REDUCING LUNG INJURY ASSOCIATED WITH LUNG TRANSPLANTATION

(71) Applicant: KAMADA LTD., Rechovot (IL)

(72) Inventor: Naveh TOV, Haifa (IL)

(73) Assignee: KAMADA LTD., Rechovot (IL)

(21) Appl. No.: 16/759,527

(22) PCT Filed: Oct. 29, 2018
(86) PCT No.: PCT/IL2018/051157

§ 371 Date: Apr. 27, 2020

Related U.S. Application Data
(60) Provisional application No. 62/579,166, filed on Oct. 31, 2017.

(51) Int. Cl. A61K 38/57; A61K 9/00

(52) U.S. Cl. CPC A61K 38/57; A61K 9/0019; A61K 9/007

(57) ABSTRACT

The present invention discloses methods for preventing or reducing lung injury associated with lung transplantation in lung transplant recipients. The method of the invention comprises the administration of improved dosage regimen of Alpha-1 Antitrypsin (AAT) for prevention of acute and/or chronic refractory rejection in lung transplant patients.

### FIELD OF THE INVENTION

The present invention relates to methods for reducing lung injury in lung transplant recipients. The method of the invention comprises the administration of improved dosage regimen of Alpha-1 Antitrypsin (AAT) for prevention of acute and/or chronic refractory rejection in lung transplant patients.

### BACKGROUND OF THE INVENTION

Despite a significant increase in the number of lung transplants performed and improvements in patient care, the primary causes of death after lung transplantation have remained static during the past decade (Yusen, Edwards et al. 2014). In the early post-operative period, primary graft dysfunction (PGD) is the major cause of morbidity and mortality (Suzuki, Cantu et al. 2013). PGD develops after transplantation in approximately 20% of all lung transplant recipients. The underlying pathogenesis of PGD is multifactorial, with ischemia-reperfusion (IR)—related processes the most common contributing factors for PGD. Severe ischaemia-reperfusion injury (IRI) has been associated with an increased risk of acute rejection and it is considered to be the main cause of primary graft failure (den Hengst, Gielis et al. 2010).

During ischemia, lactic acid, a product of anaerobic metabolism, accumulates in the tissue causing acidosis and altering enzymatic kinetics. This leads to ATP depletion, cellular damage and interstitial edema. Although cellular metabolism is reduced during cold static storage, pneumocytes in the graft are still subject to oxidative stress, intracellular electrolyte imbalance and activation of apoptotic pathways. Thoracic surgery compounds this problem as damage to the alveolar epithelium and endothelium allows the passage of high molecular weight proteins, which generates edema in the alveolar space (den Hengst, Gielis et al. 2010, Rancan, Paredes et al. 2017). Post-transplantation mechanical ventilation can further damage the pulmonary tissue by changing both pressures and volumes. This damage can trigger an inflammatory response and the activation of innate immunity and plasma cascade systems, which contribute to generate a pulmonary edema. The consequent injury has been identified as

[symbol]

a significant cause of morbidity and mortality in the early postoperative period. Following the initial edema, there is an influx of neutrophils to the area. These cells mediate tissue damage through the production of reactive oxygen intermediates (ROS), inflammatory cytokines, and destructive enzymes including neutrophil elastase.

Plasma derived AAT (pAAT) is currently used therapeutically for the treatment of pulmonary emphysema in patients who have a genetic AAT deficiency, also known as Alpha-1 Antitrypsin Deficiency or Congenital Emphysema. Purified pAAT has been approved for replacement therapy (also known as “augmentation therapy”) in these patients. There is a continuous effort targeted at producing recombinant AAT, but as of today there is no approved recombinant product. The endogenous role of AAT in the lungs is predominantly to regulate the activity of neutrophil elastase, which breaks down foreign proteins present in the lung. In the absence of sufficient quantities of AAT, the elastase breaks down lung tissue, which over time results in chronic lung tissue damage and emphysema.

Several clinical trials address the potential benefit of AAT therapy to individuals with normal AAT production (i.e. not defined as AAT deficient subjects), including islet and lung transplantation, T1DM, graft-versus-host disease, acute myocardial infarction, and cystic fibrosis. The initial dosing plan in many of these trails was taken from the long-standing protocols of AAT augmentation therapy for AAT-deficient patients.

However, the timing, dosage and duration of AAT treatment required for the preservation of graft rejection in lung transplant recipients cannot be simply extrapolated from those found to be effective in treating the genetic AAT deficiency and the disorders associated thereto.

administering AAT in a multiple-dose regimen resulted in a lower median days on mechanical ventilation and a lower median hospitalization days for the patients in the AAT treated group as compared to the patients in the control group.

According to one aspect, the present invention provides a method of treating a lung disorder, lung disease, or lung injury associated with lung transplantation in a subject in need thereof, comprising administering to the subject AAT in a multiple variable dosage regimen, thereby treating the lung disorder, lung disease, or lung injury associated with lung transplantation in said subject. According to certain embodiments, the lung disorder associated with lung transplantation is selected from the group consisting of: re-inflammation, Acute Respiratory Distress Syndrome (ARDS), inflammation, graft rejection, primary graft failure, ischemia-reperfusion injury, reperfusion injury, reperfusion edema, allograft dysfunction, acute graft dysfunction, pulmonary re-implantation response, bronchiolitis obliterans, and primary graft dysfunction (PGD).

According to certain embodiments, the lung injury associated with lung transplantation is PGD.

According to another aspect, the present invention provides a method for preventing or reducing graft rejection in a lung transplant recipient comprising administering to the recipient AAT in a multiple variable dosage regimen sufficient to prevent or reduce graft rejection. According to certain embodiments, the graft rejection is acute or chronic.

According to certain embodiments, the method of the present invention reduces the number of days under mechanical ventilation and hospitalization. [symbol] mg AAT/KgBW to about 240 mg AAT/KgBW.

According to certain embodiments, each portion dose comprises 30, 90, 120 or 240 mg AAT/KgBW. According to certain embodiments, the multiple portion doses are administered at intervals of from about 2-4 days to about 2-4 weeks. According to certain embodiments, the intervals are selected from constant intervals and variable intervals. According to certain embodiments, the multiple portion doses contain the same amount of AAT. According to certain embodiments, the multiple portion doses contain variable amounts of AAT. According to certain embodiments, the multiple portion doses are administered at intervals of two weeks.

According to certain embodiments, the amount of AAT is descending from the first dose administered to the second dose administered. According to certain embodiments, the AAT is selected from the group consisting of plasma-derived AAT and recombinant AAT. According to certain embodiments, the subject is human

Any route of administration as is known in the art to be suitable for AAT administration can be used according to the teachings of the present invention. According to certain embodiments, the AAT is administered parenterally. According to certain exemplary embodiments, the AAT is administered intravenously (i.v.). According to some embodiments, the AAT is administered via inhalation. According to some embodiments, the dosage regimen for inhalation is about 7 mg/kgBW weekly (80 mg×7 days/80 KgBW). According to other embodiments, the AAT is administered by subcutaneous administration. The AAT is typically administered within a pharmaceutical composition formulated to complement with the route of administration.

According to another aspect, the present invention provides a method for the prolonging of lung implant survival in a subject undergoing lung implantation, of the lung transplantation. According to certain exemplary embodiments, the side effects are selected from the group consisting of apoptosis, production of cytokines, production of NO, or any combinations thereof.

According to a further aspect, the present invention provides a method for delaying onset or diminishing progression of one or more complications associated with lung transplantation in a subject, the method comprising the administration of an effective amount of AAT, wherein the method can result in: reduced hospitalization; reduced intensive care or mechanical ventilation need; reduced healthcare utilization or burden; reduced absences from school or work; decreased antibiotic need; decreased steroid need; decreased morbidity; and improved quality of life for subjects.

Other objects, features and advantages of the present invention will become clear from the following description and drawings.

### BRIEF DESCRIPTION OF THE FIGURES

FIG. 1 shows the effect of AAT (Glassia) treatment on the days of mechanical ventilation that the lung transplantation patients were maintained on during the first three months of treatment as compared to the control (SOC) treated group. Circles represent patients treated with Glassia and SOC; triangles represent patients treated only with SOC.

FIG. 2 shows the effect of AAT (Glassia) treatment on the hours of index mechanical ventilation.

FIG. 3 shows the effect of AAT (Glassia) treatment on transplanted lung function as measured by PaO₂/FiO₂ ratios at Day 3.

FIG. 4 shows the percentage of patients with primary graft dysfunction (PGD) grade at [symbol] transplantation) and at the end of the treatment period (48 weeks), showed an improvement in function in patients who completed the treatment period.

FIG. 7 shows the effect of AAT (Glassia) treatment on pulmonary function tests (forced vital capacity (FVC)).

FIG. 8 shows the effect of AAT (Glassia) treatment on the 6-min walk test at 48 weeks.

FIG. 9 demonstrates that AAT reduces the neutrophils infiltration to transplanted lungs and bronchoalveolar lavage (BAL) fluids. FIG. 9A shows the counts of neutrophils in the BAL fluids of transplanted lungs. FIG. 9B shows the neutrophils infiltration in transplanted lungs.

FIG. 10 demonstrates that AAT reduces the incidence of Ischemia/Reperfusion injury in transplanted lungs.

FIG. 11 demonstrates that AAT exhibits an attenuating effect on acute rejection without immunosuppression.

FIG. 12 demonstrates that the cytokine levels (INF GAMMA) were reduced after AAT treatment, in BAL samples collected from the transplanted lung of rats at the indicated days post transplantation (n=3 per time point). Cytokine levels were detected in triplicates, using the bead-based method (Luminex).

FIG. 13 demonstrates that the cytokine levels (GCSF) were reduced after AAT treatment, in serum samples collected from rats at the indicated days post lung transplantation (n=3 per time point).

FIG. 14 demonstrates that the cytokine levels (IL-12p70) were reduced after AAT treatment, in serum samples collected from rats at the indicated days post lung

[symbol]

### DEFINITIONS

As used herein, “a” or “an” may mean one or more than one of an item.

As used herein the term “about” refers to the designated value ±10%.

As used herein, the term “Alpha-1 Antitrypsin” (AAT) refers to a glycoprotein that in nature is produced by the liver and lung epithelial cells and secreted into the circulatory system. AAT belongs to the Serine Proteinase Inhibitor (Serpin) family of proteolytic inhibitors. This glycoprotein consists of a single polypeptide chain containing one cysteine residue and 12-13% of the total molecular weight of carbohydrates. AAT has three N-glycosylation sites at asparagine residues 46, 83 and 247, which are occupied by mixtures of complex bi- and triantennary glycans. This gives rise to multiple AAT isoforms, having isoelectric point in the range of 4.0 to 5.0. The glycan monosaccharides include N-acetylglucosamine, mannose, galactose, fucose and sialic acid. AAT serves as a pseudo-substrate for elastase; elastase attacks the reactive center loop of the AAT molecule by cleaving the bond between methionine358-serine359 residues to form an AAT-elastase complex. This complex is rapidly removed from the blood circulation. AAT is also referred to as “alpha-1 Proteinase Inhibitor” (API). The term “glycoprotein” as used herein refers to a protein or peptide covalently linked to a carbohydrate. The carbohydrate may be monomeric or composed of oligosaccharides. It is to be explicitly understood that any AAT as is or will be known in the art, including plasma-derived AAT and recombinant AAT can be used according to the teachings of the present invention.

As used herein “analog of alpha-1-antitrypsin” may mean a compound having alpha-1-antitrypsin-like activity. In one embodiment, an analog of alpha-1-antitrypsin is a functional derivative of alpha-1-antitrypsin. In a particular embodiment, an analog of fragments thereof, fusion proteins or fragments of AAT, homologues obtained by analogous substitution of one or more amino acids of AAT, and species homologues. For example, the gene coding for AAT can be inserted into a mammalian gene encoding a milk whey protein in such a way that the DNA sequence is expressed in the mammary gland as described in, e.g., U.S. Pat. No. 5,322,775, which is herein incorporated by reference for its teaching of a method of producing a proteinaceous compound. “Recombinant AAT,” also refers to AAT proteins synthesized chemically by methods known in the art such as, e.g., solid-phase peptide synthesis Amino acid and nucleotide sequences for AAT and/or production of recombinant AAT are described by, e.g., U.S. Pat. Nos. 4,711,848; 4,732,973; 4,931,373; 5,079,336; 5,134,119; 5,218,091; 6,072,029; and Wright et al., Biotechnology 9: 830 (1991); and Archibald et al., Proc. Natl. Acad. Sci. (USA), 87: 5178 (1990), are each herein incorporated by reference for its teaching of AAT sequences, recombinant AAT, and/or recombinant expression of AAT.

“Acute” as used herein means arising suddenly and manifesting intense severity. With relation to delivery or exposure, “acute” refers to a relatively short duration.

“Chronic” as used herein means lasting a long time, sometimes also meaning having a low intensity. With regard to delivery or exposure, “chronic” means for a prolonged period or long-term.

The terms “prevent” or “preventing” includes alleviating, ameliorating, halting, restraining, slowing, delaying, or reversing the progression, or reducing the severity of pathological conditions described above, or forestalling the onset or development of a disease, disorder, or condition for a period of time from minutes to indefinitely. Prevent also means reducing risk of developing a disease, disorder, or condition.

“Amelioration” or “ameliorate” or “ameliorating” refers to a lessening of at least

Lung transplantation has become a treatment of choice for patients with advanced/end-stage lung diseases. Indications for lung transplantation include chronic obstructive pulmonary disease (COPD), pulmonary hypertension, cystic fibrosis, idiopathic pulmonary fibrosis, and Eisenmenger syndrome. Typically, four different surgical techniques are used: single-lung transplantation, bilateral sequential transplantation, combined heart-lung transplantation, and lobar transplantation, with the majority of organs obtained from deceased donors. Within last decades, donor management, organ preservation, immunosuppressive regimens and control of infectious complications have been substantially improved and the operative techniques of transplantation procedures have been developed. Nonetheless, primary graft dysfunction (PGD) affects an estimated 10 to 25% of lung transplants and is the leading cause of early post-transplantation morbidity and mortality for lung recipients (Lee J C and Christie J D. 2009. Proc Am Thorac Soc, vol. 6: 39-46). PGD manifests as an acute lung injury defined by diffuse infiltrates on chest x-ray and abnormal oxygenation. There, there is some evidence to suggest a relationship between reperfusion injury, acute rejection, and the subsequent development of chronic graft dysfunction. Chronic rejection, known as obliterative bronchiolitis/bronchiolitis obliterans syndrome (BOS), is the key reason why the five year survival is only 50%, which is significantly worse than most other solid organ transplants. Investigators have recently demonstrated that PGD increases the risk of the development of BOS independent of other risk factors, and the severity of PGD is directly associated with increased risk for BOS (Daud S A, Yusen R D et al. 2007 Am J Respir Crit Care Med. 2007; 175(5):507-513).

As used herein, the term “Idiopathic pulmonary fibrosis (IPF)” refers to a type of lung disease that results in scarring (fibrosis) of the lungs for an unknown reason. Over time the scarring gets worst and it becomes hard to take in a deep breath and the lungs

[symbol]

The term “emphysema,” as is used herein, refers to a pathological condition of the lungs in which there is a decrease in respiratory function and often breathlessness due to an abnormal increase in the size of the air spaces, caused by irreversible expansion of the alveoli and/or by the destruction of alveolar walls by neutrophil elastase. Emphysema is a pathological condition of the lungs marked by an abnormal increase in the size of the air spaces, resulting in strenuous breathing and an increased susceptibility to infection. It can be caused by irreversible expansion of the alveoli or by the destruction of alveolar walls. Due to the damage caused to lung tissue, elasticity of the tissue is lost, leading to trapped air in the air sacs and to impairment in the exchange of oxygen and carbon dioxide. In light of the walls breakdown, the airway support is lost, leading to obstruction in the airflow. Emphysema and chronic bronchitis frequently co-exist together to comprise chronic obstructive pulmonary disease.

As used herein, the term “chronic obstructive pulmonary disease” abbreviated “COPD”, refers to a disease state characterized by airflow limitation that is not fully reversible. The airflow limitation is usually both progressive and associated with an abnormal inflammatory response of the lungs to noxious particles or gases. COPD is the fourth leading cause of death in America, claiming the lives of 120,000 Americans in 2002, with smoking being a primary risk factor. A diagnosis of COPD exacerbation is considered when there is increases dyspnea, increased sputum volume, and increased sputum purulence. Severity of an exacerbation can be quantified by assessing the magnitude of these three symptoms (Dewan N A 2002. Chest 122:1118-1121).

“Bronchiectasis,” as used herein, refers to the abnormal and irreversible dilation of the proximal medium-sized bronchi (>2 mm in diameter) caused by destruction of the muscular and elastic components of the bronchial walls. It can be congenital or [symbol] produces sputum and mucus, for at least three months in two consecutive years. Mucous gland enlargement is the histologic hallmark of chronic bronchitis. The structural changes described in the airways include atrophy, focal squamous metaplasia, ciliary abnormalities, variable amounts of airway smooth muscle hyperplasia, inflammation, and bronchial wall thickening Neutrophilia develops in the airway lumen, and neutrophilic infiltrates accumulate in the submucosa. The respiratory bronchioles display a mononuclear inflammatory process, lumen occlusion by mucous plugging, goblet cell metaplasia, smooth muscle hyperplasia, and distortion due to fibrosis. These changes, combined with loss of supporting alveolar attachments, cause airflow limitation by allowing airway walls to deform and narrow the airway lumen.

The term “dosage” as used herein refers to the amount, frequency and duration of AAT which is given to a subject during a therapeutic period.

The term “dose” as used herein, refers to an amount of AAT which is given to a subject in a single administration.

The terms “multiple-variable dosage” and “multiple dosage” are used herein interchangeably and include different doses of AAT administration to a subject and/or variable frequency of administration of the AAT for therapeutic treatment. “Multiple dose regimen” or “multiple-variable dose regimen” describe a therapy schedule which is based on administering different amounts of AAT at various time points throughout the course of therapy.

The term “total cumulative dose” as used herein, refers to the total amount of a drug given to a patient over time.

“Inhalation” refers to a method of administration of a compound that delivers an [symbol] patient by inhalation through the mouth and into the lungs.

The term “dry powder” refers to a powder composition that contains finely dispersed dry particles that are capable of being dispersed in an inhalation device and subsequently inhaled by a subject.

### Pharmaceutical Compositions

According to certain embodiments, AAT is administered in the form of a pharmaceutical composition. As used herein, the term “pharmaceutical composition” refers to a preparation of AAT with other chemical components such as pharmaceutically acceptable carriers and excipients. The purpose of a pharmaceutical composition is to facilitate administration of an active ingredient to an organism and enhance its stability and turnover.

Any available AAT as is known in the art, including plasma-derived AAT and recombinant AAT can be used according to the teachings of the present invention. According to certain exemplary embodiments, the AAT is produced by the method described in U.S. Pat. No. 7,879,800 to the Applicant of the present invention.

The term “pharmaceutically acceptable” means approved by a regulatory agency of the Federal or a state government or listed in the U. S. Pharmacopeia or other generally recognized pharmacopeia for use in animals, and more particularly in humans.

The term “carrier” refers to a diluent or vehicle that does not cause significant irritation to an organism and does not abrogate the biological activity and properties of the administered compound. An adjuvant is included under these phrases. Such pharmaceutical carriers can be sterile liquids, such as water and oils, including those of petroleum, animal, vegetable or synthetic origin, such as peanut oil, soybean oil, pharmaceutical composition to further facilitate administration of an active ingredient. Suitable pharmaceutical excipients include starch, glucose, lactose, sucrose, trehalose, gelatin, malt, rice, flour, chalk, silica gel, sodium stearate, glycerol monostearate, talc, sodium chloride, dried skim milk, glycerol, propylene glycol, water, lipids, phospholipids, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

The pharmaceutical compositions of the present invention can be manufactured by processes well known in the art, e.g., by means of conventional mixing, dissolving, granulating, dragee-making, levigating, emulsifying, encapsulating, entrapping, spray drying or lyophilizing processes.

According to certain exemplary embodiments, pharmaceutical compositions, which contain AAT as an active ingredient, are prepared as injectable, either as liquid solutions or suspensions, however, solid forms, which can be suspended or solubilized prior to injection, can also be prepared. According to additional exemplary embodiments the AAT-containing pharmaceutical composition is formulated in a form suitable for inhalation. According to yet additional embodiments, the AAT-containing pharmaceutical composition is formulated in a form suitable for subcutaneous administration. Subcutaneous administration may be a preferred mode of administration, because administration of AAT at multiple low doses was shown to have a positive effect on islet protection. From the patient point of view multiple [symbol] include, but are not limited to, intravenous, subcutaneous, intramuscular, intraperitoneal, oral, topical, intradermal, transdermal, intranasal, epidural, ophthalmic, vaginal and rectal routes. The pharmaceutical compositions can be administered by any convenient route, for example by infusion or bolus injection, by absorption through epithelial linings (e.g., oral mucosa, rectal and intestinal mucosa, etc.), and may be administered together with other therapeutically active agents. The administration may be localized, or may be systemic. Pulmonary administration can also be employed, e.g., by use of any type of inhaler or nebulizer.

Pharmaceutical compositions for use in accordance with the present invention may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, typically in physiologically compatible buffers such as Hank's solution, Ringer's solution, or physiological salt buffer. For transmucosal or transdermal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules after adding suitable auxiliaries as desired to obtain tablets or

[symbol]

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for administration should be in dosages suitable for the chosen route of administration.

For buccal administration, the compositions may take the form of tablets or lozenges formulated in conventional manner

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, traditional binders and carriers such as triglycerides, microcrystalline cellulose, gum tragacanth or gelatin.

According to certain exemplary embodiments, the AAT-containing pharmaceutical composition used according to the teachings of the present invention is a ready-to-use solution. According to further exemplary embodiments the AAT-containing pharmaceutical composition is marketed under the trade name Glassia®.

Therapeutic Methods

In one embodiment of the present invention, methods provide for treating a subject in need of or undergoing lung transplantation. For example, treatments for reducing graft rejection, promoting graft survival, and promoting prolonged graft function by administering to a subject in need thereof a therapeutically effective amount of a composition. The composition can include a compound capable of inhibiting at least one serine protease for example, alpha 1-antitrypsin, or analog thereof. [symbol] invention include reducing negative effects on lung during explant, isolation, transport and/or prior to implantation. For example, the composition can reduce apoptosis, reduce production of cytokines, reduce production of NO, or combination thereof in the lung for transplant. In one particular embodiment, a composition can include a compound that includes alpha-1-antitrypsin, an analog thereof, a serine protease inhibitor, serine protease inhibitor-like activity, analog thereof or a combination thereof.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Phase II Study to Evaluate the Safety and Efficacy of Intravenous AAT Treatment in Lung Transplantation

### Objectives:

1. To assess the safety of AAT administration in subjects undergoing first lung transplantation.
2. To assess the effect of AAT administration on rate and severity of acute and chronic lung rejection as well as pulmonary infections in subjects undergoing first lung transplantation.

Number of Subjects:

Approximately 30 lung transplant candidates were randomized to receive either AAT therapy in addition to standard of care (SOC) or SOC only. Subjects were randomized 2:1 to the treatment arm or SOC only, respectively.

Inclusion Criteria: Age ≥18 years, [symbol]
a. History of IgA deficiency (undetectable levels) and anti IgA antibodies
b. In case IgA levels that are detectable but below lab normal range, the investigator should conduct to sponsor's medical director for approval to include the subject.
2. Known history for hepatitis B virus (HBV), hepatitis C virus (HCV) or human immunodeficiency virus (HIV) Type 1/2 infection.
3. Subjects with a history of severe immediate hypersensitivity reactions, including allergies, anaphylaxis to plasma products or any human proteins of different source.
4. Pregnant or lactating women at entry to study and women of child bearing potential, who are unwilling to agree to continue to use acceptable methods of contraception throughout the study.
5. Presence of psychiatric/mental disorder or any other medical disorder which might impair the subject's ability to give informed consent or to comply with the requirements of the study protocol.
6. Alcohol abuse or history of alcohol abuse.
7. Illegal drugs.
8. Candidate for organ transplantation other than first lung or heart-lung transplantation.
9. Clinically significant bronchial stenosis unresponsive to dilation and/or stenting
10. Participation in another interventional clinical trial within 30 days prior to baseline visit.
11. Inability to attend scheduled clinic visits and/or comply with the study protocol.

### Investigational Product, Dosage and Mode of Administration:

GLASSIA® is presented as a 50 ml solution containing 2% of active Alpha-1 antitrypsin (AAT) in a phosphate-buffered saline solution. Route of administration:

### Duration of Treatment:

The first AAT treatment will be given as close as possible to the surgical time and within following timelines window: up to 12 hours before the start time of the lung transplantation surgery or 12 hours following the stop time of the surgery. Additional administration of AAT will be given during one year after transplantation, in different intervals and doses, as per the Table 2:

TABLE 2

| AAT | | Dose |
|---|---|---|
| administration # | Time of administration | mg/kg |
| 1 | 0 day (stop time of the lung | 90 |
| | transplantation surgery) | |
| 2 | 60 hours (±22 hour) after | 30 |
| | transplantation | |
| 3 | 5th day | 30 |
| 4 | 7th day | 30 |
| 5 | 9th day | 30 |
| 6 | 11th day | 30 |
| 7 | 14th day | 120 |
| 8 | Week 4 | 120 |
| 9 | Week 6 | 120 |
| 10 | Week 8 | 240 |
| 11 | Week 12 (~Month 3) | 240 |
| 12 | Week 16 (~Month 4) | 240 |
| 13 | Week 20 (~Month 5) | 240 |
| 14 | Week 24 (~Month 6) | 240 |
| 15 | Week 28 (~Month 7) | 240 |
| 16 | Week 32 (~Month 8) | 240 |
| 17 | Week 36 (~Month 9) | 240 |
| 18 | Week 40 (~Month 10) | 240 |
| 19 | Week 44 (~Month 11) | 240 |
| 20 | Week 48 (~Month 12) | 240 |
| Follow up (FU) | Weeks 60-96 | — |

Therapy for prevention and treatment of osteoporosis: calcium and vitamin D supplementation
Therapy for prevention developing cytomegalovirus (CMV): valganciclovir hydrochloride (Valcyte®)

Each subject will participate in study for approximately 96 weeks (48 weeks of dosing and 48 weeks of FU). This study is expected to last approximately 192 weeks (42 months) (first visit of the first subject to last visit of the last subject).

### Interim Analysis Population Patients

The analysis presented here is on the first 90 days in the study of the 30 first patients randomized, but without one patient in the Glassia® plus SOC population who died before receiving study medication. This patient population was termed the “Interim Analysis” population.

### Demographic Characteristics

The smoking history is shown below but all were non-smokers with at least one pack free year at the time of transplant.

TABLE 3
Demography of Interim Analysis Population
Treatment Arm

| | | | | |
|---|---|---|---|---|
| | Glassia ® plus SOC | SOC | Total | |
| Parameter | N = 19 | N = 10 | N = 29 | P-value |

Age

| Mean ± SD | 58.4 ± 123 | | 58.5 ± 12.1 | | 58.5 ± 12.0 | | 0.8364 |
|---|---|---|---|---|---|---|---|
| Median (range) | 63.0 | (28.5-69.9) | 61.0 | (38.7-72.9) | 62.9 | (28.5-72.9) | |

| | | | | |
|---|---|---|---|---|
| Gender M:F | 17:2 | 8:2 | 25:4 | 0.5920 |
| BMI (N) | 19 | 9 | 28 | |

Etiology*. Main reason for Lung Transplant N (%)

| | | | | | | |
|---|---|---|---|---|---|---|
| IPF | 13 | (68.4) | 5 | (50) | 18 | (62.1) |
| COPD | 3 | (15.8) | 2 | (20) | 5 | (17.2) |

| | | | | | |
|---|---|---|---|---|---|
| Chronic | 0 | 1 | (10) | 1 | (3.5) |
| Obstructive | | | | | |
| Pulmonary | | | | | |
| Fibrosis | | | | | |

| | | | | | |
|---|---|---|---|---|---|
| CF | 2 | (10.5) | 0 | 2 | (6.9) |
| Pulmonary | 1 | (5.3) | 0 | 1 | (3.5) |
| Hypertension | | | | | |

| | | | | | |
|---|---|---|---|---|---|
| Sarcoidosis | 0 | 1 | (10) | 1 | (3.5) |
| Bronchiectasis | 0 | 1 | (10) | 1 | (3.5) |

| | | | |
|---|---|---|---|
| Transplant Type | 6:4 | 3:3 | 9:7 |
| Single L:R | | | |
| Double | 9 | 4 | 13 |

Pulmonary Function Tests
% FEV1

| Mean ± SD | 39.6 ± 14.7 | | 35.4 ± 15.7 | | 38.3 ± 14.8 | | 0.4600 |
|---|---|---|---|---|---|---|---|
| Median (range) | 41 | (15-65) | 32 | (15-62) | 39 | (15-65) | |

FEV1

| Mean ± SD | 1.2 ± 0.4 | | 1.1 ± 0.4 | | 1.2 ± 0.4 | | 0.5382 |
|---|---|---|---|---|---|---|---|
| Median (range) | 1.2 | (0.5-2.1) | 1.2 | (0.6-1.5) | 1.2 | (0.5-2.1) | |

Smoking History N (%)

| | | | | | | | |
|---|---|---|---|---|---|---|---|
| Ex-Smoker | 13 | (68.4) | 7 | (70) | 20 | (69) | 1.0000 |
| Never smoked | 6 | (31.6) | 3 | (30) | 9 | (31.3) | |

*IPF = Idiopathic Pulmonary Fibrosis, COPD = Chronic obstructive pulmonary disease, CF = cystic

[symbol]

[symbol]

indicates data missing or illegible when filed

### Mechanical Ventilation and Hospital Stay Days are Given as Median and Range

Four patients were recorded as experiencing primary graft dysfunction. Three (207, 209, and 219) were from the Glassia® arm and one (208) from the SOC arm. In the case of the first two Glassia® patients, the event was resolved after 11 and 7 days respectively, however in the last patient, there have been repeated infections and he remains ventilated. In the case of the SOC arm patient, 208, the event escalated and required the patient to be connected to an ECMO. A chest scan showed bilateral pulmonary edema and atelectasis of the left lung. Resistant Acinetobacter, Klebsiella, and Aspergillus, identified as originating from the donor lung were cultured from the sputum. He was treated for the edema and given antibiotics for the pulmonary infection but despite maximal treatment his condition deteriorated with renal failure, gastrointestinal hemorrhage, septic shock and multi organ failure. He died 25 days after the lung transplant.

### Days on Mechanical Ventilation

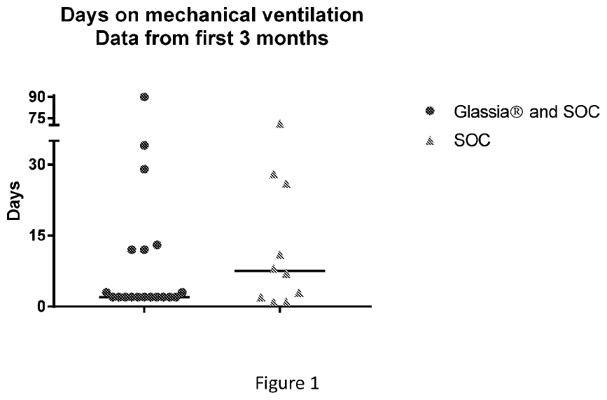

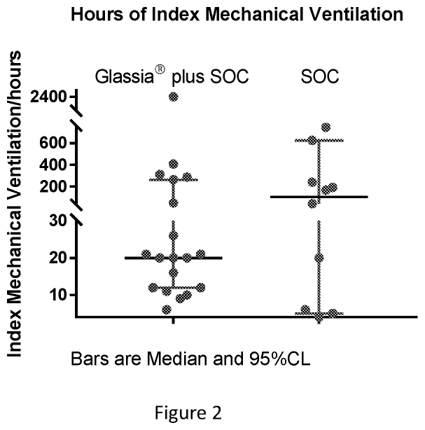

Considering the initial ventilation, the median days on mechanical ventilation was lower on AAT plus SOC than on SOC (2 days on AAT and 5 days on SOC). FIG. 1 approach of looking at the durations of individual patients within the populations the proportions of patients in each arm above and below an arbitrary duration of ventilation can provide an interesting comparison. In the Glassia arm 68% (13/19) patients required mechanical ventilation for fewer than 30 hours; this may be compared with the 40% (4/10) of patients in the control arm.

### Pulmonary Arterial Oxygen Level

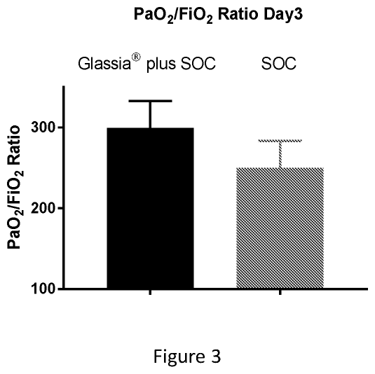

The ratios of partial pressure arterial oxygen and fraction of inspired oxygen (PaO₂/FiO₂) were determined in all patients at Day 3, the data are shown in FIG. 3. The visual impression of the comparative distribution of the data points in each arm suggest better lung function (with a higher PaO₂/FiO₂ ratio) at Day 3 in the Glassia arm.

### PGD Assessment

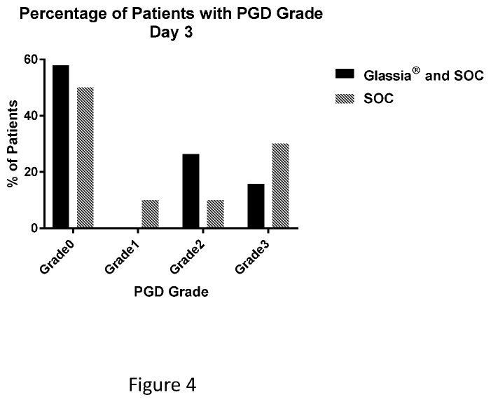

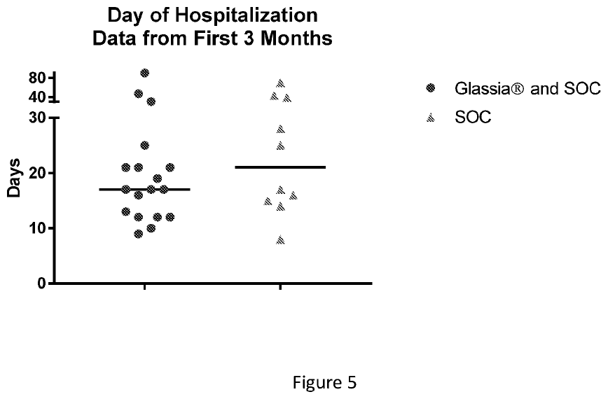

PGD scores were derived using the 2005 consensus grading system and the results are shown in FIG. 4. The proportions of the numbers of patients with grade 3 PGD compared to the numbers of patients with grade 0 PGD are of interest. In the Glassia arm, 12 patients have grades 1-3 PGD and 7 patients have grades 0 PGD at day 0: 37% of patients have grade 0 PGD. In the control arm the percentage of patients with grade 0 GHD is 10%. At day 3 the Glassia arm has 63% of patients with grade 0 PGD compared to 50% of control patients.

### Days of Hospitalization

The median hospitalization days was 17 days in the AAT plus SOC group versus 22.5 days in the SOC group. Patients in the AAT+SOC arm tended to spend fewer days on mechanical ventilation post-operatively. FIG. 5 demonstrates the effect of AAT (Glassia) treatment on the days that the lung transplantation patients were oxygen levels, PGD grade (using the 2005 criteria) and the duration of mechanical ventilation support post-transplantation. The intermediate term consequences on pulmonary function have also been evaluated.

### Pulmonary Function Tests

Considering the FEV₁ levels at screening, a baseline value (4-6 weeks after transplantation) and at the end of the treatment period (48 weeks), showed an improvement in function in patients who completed the treatment period (FIG. 6).

There was an improvement after transplantation, which was maintained with slight improvement over the 48 weeks of treatment. While there was no significant difference between the groups at screening or at 48 weeks (p=0.4488 and p=0.3446 respectively by Mann Whitney test), at 4-6 weeks there was a significant difference in favour of the Glassia plus SOC group (p=0.0427 one tailed Mann Whitney test).

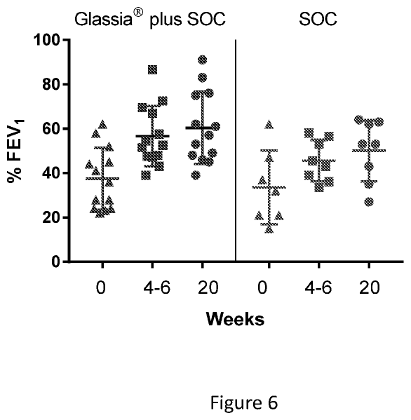

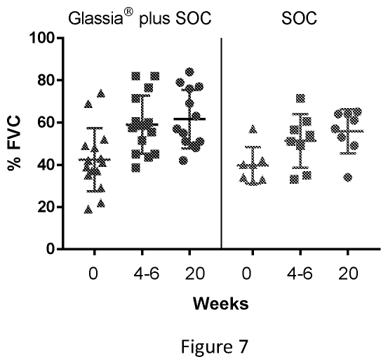

FIG. 7 shows the effect of AAT (Glassia) treatment on pulmonary function tests (forced vital capacity (FVC)). The percentage of FVC is higher after 4-6 and 20 weeks of treatment in the Glassia plus SOC group as compared to the control group.

### 6-Min Walk Test

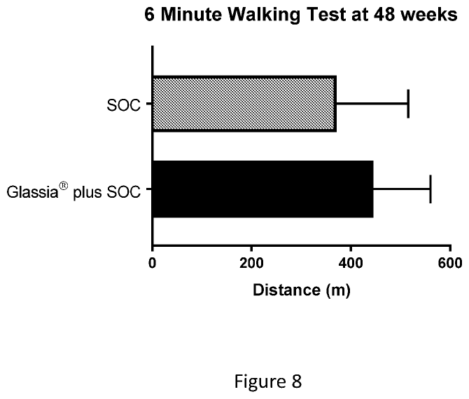

The 6-min walk test (6 MWT) is a submaximal exercise test measures the distance walked over a span of 6 minutes. The test provides information about the functional capacity, response to therapy and prognosis across a broad range of chronic cardiopulmonary conditions. As demonstrated in FIG. 8, in the Glassia arm the patients showed a better performance as compared to the control arm.

[symbol]

rejection), neutrophil counts in BAL samples and immunohistochemistry of neutrophil infiltrates in transplanted lungs. Cytokines levels were detected in BAL of transplant lungs and in serum.

### Results

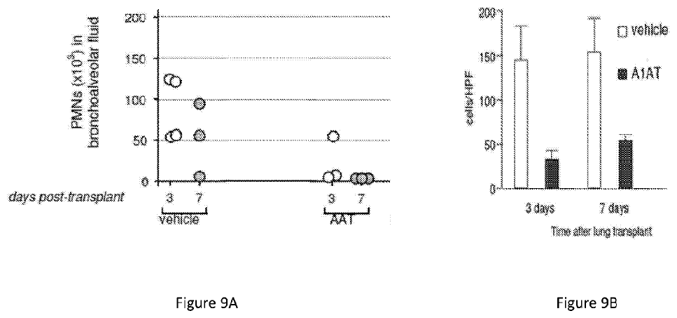

Neutrophil counts in the BAL samples of transplanted lungs from all experimental rats indicated that AAT reduced the counts of BAL neutrophils at day 3 and 7 post-transplant, compared to cells found in BAL from rats given vehicle (FIG. 9A).

Neutrophil infiltrates in the transplanted lung were evaluated with immunofluorescence technique using the His48 anti-neutrophil specific antibody at 3 and 7 days post-transplant. Results presented in FIG. 9B show a reduction of infiltrates in animals treated with AAT. The number of neutrophils found in the lung of transplanted animals treated with AAT is about 3 to 5-fold lower than the vehicle treated group.

Histopathological evaluation showed that about 50% (5/10) of transplanted rats given vehicle developed the characteristic lesions of IRI during the first 10 days post-transplant, while only 11% (1/9) rats given AAT developed IRI lesions. (Table 5). During the total 15 experiment days 42% (5/12) versus 17% (2/12) rats developed IRI lesions in the vehicle and AAT-treated groups, respectively.

TABLE 5
Ischemia/reperfusion injury frequency is expressed as ratio between
number of transplanted lungs where IRI was assessed and total number
of gradable lungs. Gradable lungs are transplanted lungs with a conserved
structure where histological evaluation was possible.

| Time from Transplant | Vehicle | AAT |
|---|---|---|
| [symbol] | [symbol] | [symbol] |
| Incidence lungs with IRI | 50% | 11% |
| Total (all time points) | 5/12 | 2/12 |
| Incidence lungs with IRI | 42% | 17% |

[symbol]

indicates data missing or illegible when filed

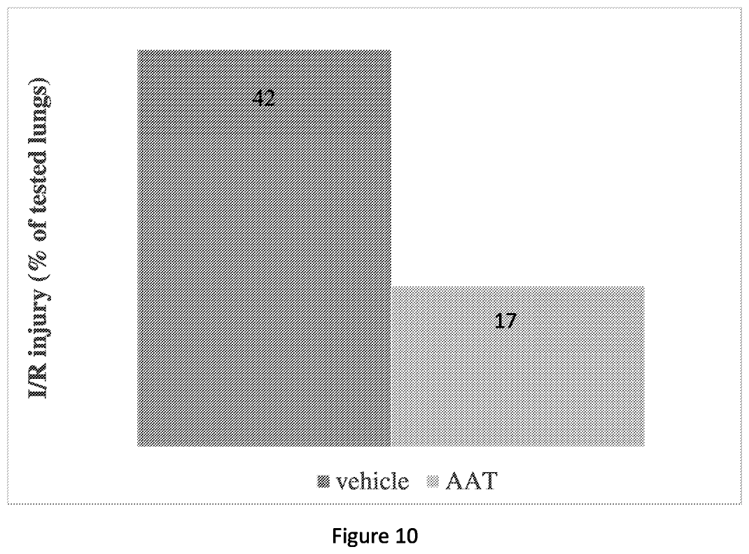

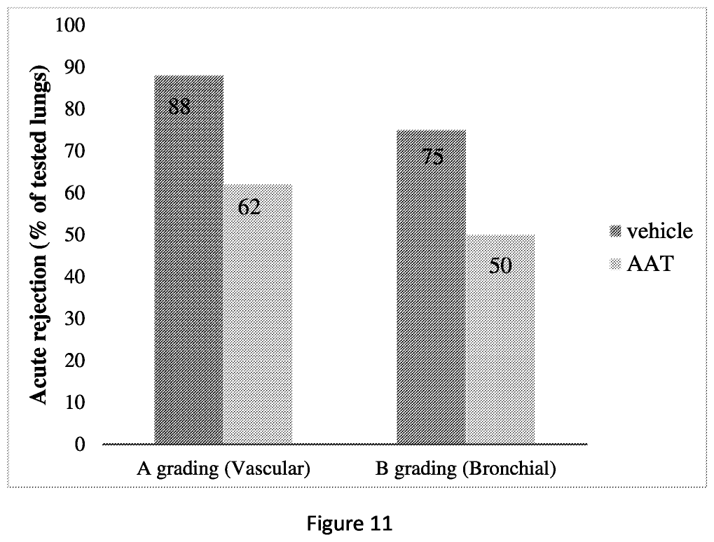

As shown in FIG. 10, AAT reduces the incidence of Ischemia/Reperfusion injury in transplanted lungs. As shown in FIG. 11, AAT exhibits an attenuating effect on acute rejection without immunosuppression.

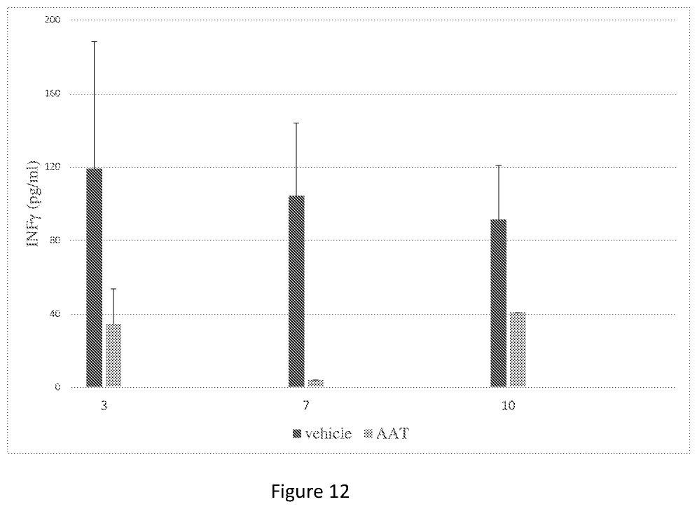

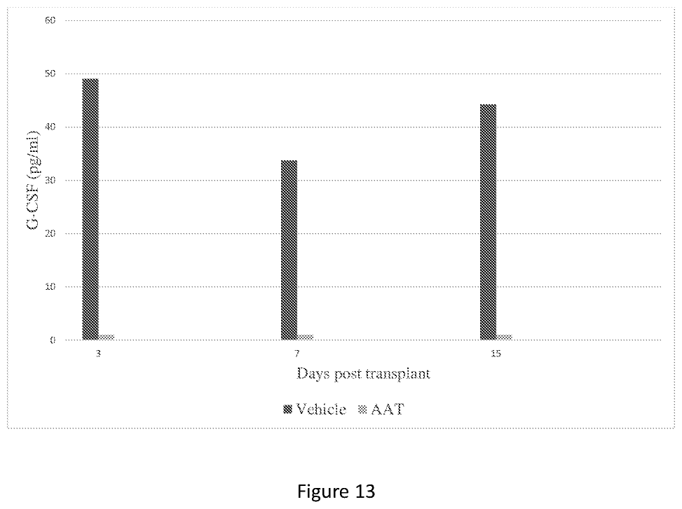

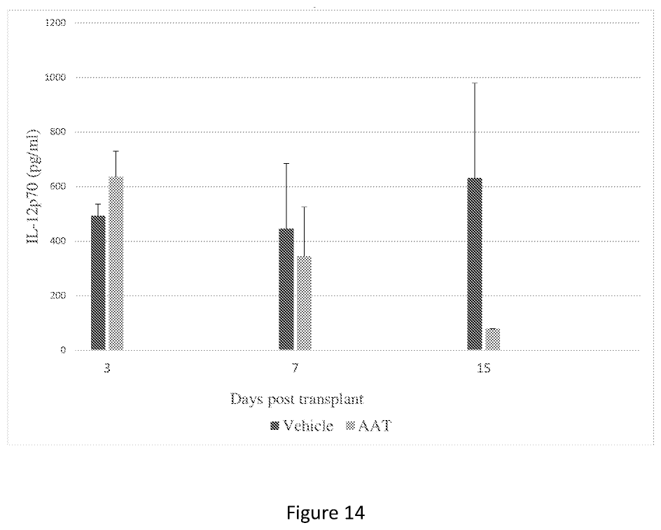

FIG. 12 demonstrates the Interferon gamma (IFNγ) levels in BAL samples collected from the transplanted lung of rats at days 3, 7 and 10 post transplantation (n=3 per time point). Cytokine levels were detected in triplicates, using the bead-based method (Luminex). Rats treated with AAT showed reduced levels of IFNγ compared to rats treated with vehicle. As shown in FIG. 13, rats treated with AAT showed reduced levels of G-CSF compared to rats treated with vehicle. As shown in FIG. 14, rats treated with AAT also showed reduced levels IL-12p70.

These results support the non-protease inhibitory actions of AAT which affect cells of the innate compartment of the immune system. Common cytokine responses under AAT treatment include a reduction in levels of the pro-inflammatory cytokines. In addition, the data demonstrate the reduction of Th1-related cytokines (INFγ and IL-12p′70) and of the systemic cytokine G-CSF that induces proliferation and maturation of pre-neutrophils to mature neutrophils.

The foregoing description of the specific embodiments will so fully reveal the general nature of the invention that others can, by applying current knowledge, readily

1. A method for preventing or reducing lung injury associated with lung transplantation in a lung transplant recipient in need thereof, the method comprising administering to the recipient AAT in a multiple variable dosage regimen sufficient to prevent or reduce the lung injury.

2. The method of claim 1, wherein the recipient is scheduled to undergo first lung transplantation.

3. The method of claim 1, wherein the multiple variable dosage regimen comprises administering AAT at a total cumulative dose selected from the group consisting of 120, 150, 360, 720, 960, 1000, 1500 and 3000 mg/KgBW.

4. The method of claim 3, wherein the multiple variable dosage regimen comprises administration of two doses.

5. The method of claim 1, wherein the length of the regimen is from about 1 to about 360 days.

6. The method of claim 1, wherein each individual dose comprises from about 30 mg AAT/KgBW to about 240 mg AAT/KgBW.

7. The method of claim 6, wherein each individual dose comprises 30, 90, 120 or 240 mg AAT/KgBW.

8. The method of claim 3 wherein the individual doses are administered at intervals of from about 2-4 days to about 2-4 weeks.

9. The method of claim 8, wherein the intervals are selected from constant intervals and variable intervals.

10. The method of claim 3, wherein the multiple portion doses contain the same amount of AAT.

11. The method of claim 3, wherein the multiple portion doses contain variable amounts of AAT.

12. The method of claim 3, wherein the multiple portion doses are administered at intervals of two weeks.

13. The method of claim 1, wherein the amount of AAT is descending from the first dose administered to the second dose administered.

14. The method of claim 1, wherein the lung injury associated with lung transplantation is selected from the group consisting of reinflammation, acute respiratory distress syndrome (ARDS), graft rejection, primary graft failure, ischemia-reperfusion injury, reperfusion injury, reperfusion edema, allograft dysfunction, acute graft dysfunction, pulmonary reimplantation response, bronchiolitis obliterans, and primary graft dysfunction (PGD).

15. The method of claim 1, wherein the AAT is selected from the group consisting of plasma-derived AAT and recombinant AAT.

16. The method of claim 1, wherein the AAT is administered within a pharmaceutical composition.

17. The method of claim 16, wherein the AAT is administered intravenously.

18. The method of claim 16, wherein the AAT is administered via inhalation.

19. The method of claim 18, wherein each individual dose comprises about 7 mg/kgBW AAT and is administered weekly.

20.-23. (canceled)

24. The method of claim 14, wherein the lung injury associated with lung transplantation is PGD.
